(19) United States
(12) Patent Application Publication
(10) Pub. No.: US 2007/0299040 A1
(43) Pub. Date: Dec. 27, 2007

(54) Composition for Preventing a Cold

(75) Inventor: Ji-Won Yoon, North Chicago, IL (US)

Correspondence Address: IPLA P.A., 3580 WILSHIRE BLVD., 17TH FLOOR, LOS ANGELES, CA, 90010, US

(73) Assignee: BIOTECH INSTITUTE FOR INTERNATIONAL INNOVATION, INC., KYEONGGI-DO (KR)

(21) Appl. No.: 10/598,159

(22) PCT Filed: Feb. 17, 2005
(86) PCT No.: PCT/KR05/00445

§ 371 Date: Aug. 22, 2007

(30) Foreign Application Priority Data
Feb. 18, 2004 (KR) 10-2004-0010802

(51) Int. Cl. A61K 31/59; A61K 31/352; A61K 31/355; A61P 43/00; A61K 31/51

(52) U.S. Cl. 514/167; 514/276; 514/456; 514/458

(57) ABSTRACT

The present invention relates a composition for preventing cold comprising vitamin C and ginkgetin, a use of the composition for preparing a medicine To prevent cold, a method for preventing cold by using the composition, and method for preparing cold prophylactic. The composition of the present invention has excellent inhibition effect of cold viruses such as influenza viruses, coronaviruses, rhinoviruses an adenoviruses, can remarkably increase immunity, and may be used as health care products for preventing cold or cold medicine without cytotoxicity.

### TECHNICAL FIELD

The present invention relates to a composition for preventing cold comprising vitamin C and ginkgetin, a use of the composition of preparing a medicine for preventing cold, a method for preventing cold by using the composition, and a method for preparing cold prophylactic.

### BACKGROUND ART

One cause of the most dangerous contagious disease to human beings is viruses whose size, shape, chemical composition, and effect to host are diverse. By now, antiviral agents effective for treatment and prophylaxis have been discovered only for hepatitis B, influenza A, B, HIV and the like. Antiviral agents are highly toxic to host, and so they are used locally. Thus, antiviral agents which are antitoxic to host and effective for various viruses have been needed, but no such agent exists yet.

Cold is a disease caused by the most common virus to human beings. Cold requires the most frequent treatment and care, and 25˜33% of patients treated in general hospital suffer from acute upper respiratory infection, namely cold. Cold symptoms include runny nose, cough, fever, chill, muscular pain, and headache, caused by inflammatory response in the mucosa of upper respiratory tract. Each year, 10˜15% of adults around the world or 300 million peoples are suffering from cold, and the economic loss thereby is known to be more than a billion dollars. Moreover, cold is known to be caused by about two hundred kinds of viruses such as rhinovirus, influenza virus, adenovirus, respiratory exanthema virus, enterovirus, coronavirus, coxsackievirus, echovirus, orthomyxovirus, paramyxovirus, etc. Diseases by secondary infection are more serious than cold itself. In particular, airway infection of children is very serious and may threaten their lives. Thus, cold is considered the most important cause for deaths from pulmonary diseases by airway infection among children of 5 years old and under. On the other hand, it is known that the cause of 30˜50% of adult cold is not clear, and that of the others is the same as that of children.

Of the various causes of cold, cold from influenza virus infection results in chill accompanied by high fever, myalgia, inertia, respiratory pain, headache, and abdominal pain, and its cause virus is Groups A, B and C of myxoviruses. These influenza viruses cause similar symptoms, but have completely different antigen property. Prevalence of influenza virus is periodic worldwide, and so the period of prevalence is 2-3 years in type A of influenza and 4-5 years in type B of influenza. Influenza is one of several viruses preventable by modern medical science, but the vaccine's effect has been very low. At present, influenza vaccine is effective for types A and B of viruses, but the prevention rate is merely 60˜70%. The standard vaccine to new strains should be newly prepared every year, but the period to maintain the immunity is very short. Drugs effective for the prevention and treatment of influenza are amantadin hydrochloride and rimantadin hydrochloride. However, it has been reported that these drugs are only effective for type A of influenza virus, and their efficiency is very low in case of serious infection.

Rhinoviruses cause 30˜35% of adult cold, and are the most active in early fall, spring, and summer. So far, 110 species of their strains are discovered. Coronaviruses are known to cause most adult colds. They are the cause of cold developed from winter to spring. About 30 kinds of their strains are discovered, and about three fourths (¾) of those strains are known to infect human beings. Since coronaviruses are difficult to incubate in a lab for observation, their characteristics and toxicity are less well known than those of the rhinoviruses.

To prevent viral related infections, vaccine administration is generally used. However, it is in fact impossible to prepare vaccines for all viral related diseases since each virus has its own specific antigen inducing production of specific protective protein in the human body. So far, vaccine development against various types of virus antigens has not succeeded, and preparation of effective vaccines has become more difficult due to the discovery of various mutants every year.

Therefore, the best prevention against viral related diseases is to boost the body immunity and to increase the antigen-antibody response. Consequently, the improvement of immunity may be a good means for the prevention and treatment of cold. In the human body, the immune response to viral invasion is performed by the T cells which block the proliferation of viruses and thereby remove viruses by killing virus-infected cells.

The immune system, as a natural resistance, may be divided into nonspecific immune system and specific immune system. Natural resistance indicates anatomic and physiologic elements that block all intruders breaking into the human body. The nonspecific immune system consists of phagocytes to remove intruders that have gotten through the natural resistance, while the specific immune system consists of lymphocytes. The specific immune system has the ability to remember and distinguish self from non-self. Leukocytes erect secondary and tertiary defense lines for foreign materials that have entered the body by breaking through first defense line. Among them, phagocyte has many lysosomes, containing acidic hydrolase and peroxidase, to actively ingurgitate microbes or tumor cells. These cells have cytokine receptors such as INF-γ et al. They produce cytokines such as complement component, interferon, interleukin-1, and tumor necrosis factor, and their function may be enhanced by various cytokines produced by the T-cell. Also, the T-cell, a kind of leukocyte, accounts for about 70% of small lymphocytes in the blood. It is differentiated in the thymus, and has a TCR, T-cell antigen receptor. Peripheral blood T-cell is classified into CD4 positive Th-cell (helper T-cell) and CD8 positive Tc-cell (cytotoxic T-cell). CD4+Th cell is activated by recognizing antigens bound to MHC class II molecules, and helps either B cells to produce antibodies or other T cells to function. CD4+ Th cell can also be divided into Th1 and Th2 in accordance with specific cytokines produced. In a lab mouse, Th1 cell secretes interleukin-2, INF-gamma et al., while Th2 cell secretes IL-4, 5, 6, 9, 10, 13 et al. However, in human beings, production of IL-2, 6, 10, 13 et al. is not strictly divided. Besides, IL-3, tumor necrosis factor (TNF-α), GM-CSF (granulocyte-macrophage colony stimulating factor), Metenkephalin, chemokine, etc. are commonly secreted regardless of cell type. Th1 cell gets involved in cell immune response, and activates cytotoxicity and inflammatory response. Cytokines produced in Th2 cell promote the formation of antibodies, especially helping to produce IgE, and enhance the growth and function of eosinocyte. Thus, Th2 cytokine is commonly discovered in the antibody production and allergy response. Also, it has been discovered that each of Th1 and Th2 cytokine has an inhibiting function, and anti-IL-4 antibody and anti-INF-γ antibody can change disease progress in animal infections. Moreover, a case showed that INF-γ injection improved symptoms of rheumatism patients. In short, agents which can produce IL-2 or INF-γ and the like very well are expected to have improved the effects of immune responses, thereby helping to prevent cold or treat an early onset of cold.

Also, injuries to the major histocompatibility gene complex (MHC) of T cells by nascent oxygen, thereby making T cells incapable of attacking virus effectively is another cause of reduced immunity.

Therefore, since immunological response can defend the body from various diseases, vaccination, the use of antitoxin, etc. have been tried to prevent and treat diseases by reinforcing immunological responses. Recently, direct use of immunoregulatory agents as a means for controlling immunological responses has been on the rise. Immunoregulatory agents enhance the biological defensive power against causes of diseases by enhancing immunological response of the body through nonspecifically stimulating immune cells. Such immunoregulatory agents include chemosynthetic agents, microorganism compositions, biologics and the like. Immunoregulatory agent currently in the market may induce such side effects as allergic response, defect of central nerve system, apepsia, gagging, vomiting, anorexia, diarrhea et al.

Therefore, it is important to develop an immune enhancer that can enhance the immunity without any adverse side effects to the human body. Recently, immunoregulatory agent study has been conducted by testing efficacy of harmless food material, effective component extracted from natural substance, existing Chinese medicine, etc. Particularly, physiologically active materials enhancing biogenic regulation and biogenic defense system have been actively sought from natural substances, and they have slowed gained entry as therapeutics of alternative medicine or health care products. At present, the alternative medicine market for the prevention and treatment of diseases by viruses is rapidly growing, and it has been reported that ⅓ of the U.S. population depend on alternative medicine rather than on vaccine and chemical medicine. In Europe, it is reported that the ratio is even greater. Considering this trend, development of medicine or food composition effective for enhancing immunologic function as alternative medicine appears to be necessary.

Among the essential ingredients of the present invention, vitamin C is an ascorbic acid, similar to hexose but has endiol group, and is a kind of carbohydrate synthesized from carbohydrate precursor such as glucose, galactose, etc. The aqueous solution of vitamin C is acidic, has powerful deoxidization effect enough to affect the formation and interception of harmful oxygen, and is the most unstable in the aqueous solution state. Vitamin C is distributed widely in adrenal, retina, etc., and substantial quantity is found in liver, spleen, bone marrow, pancreas, thymus, cerebrum, pituitary gland, and kidney. Vitamin C plays an important role in synthesizing collagen protein by participating in hydration reaction of praline in the human body, is involved in the metabolism of phenylalanine and tyrosine, and can help to produce adenocortical hormone. Besides, it is effective for arteriosclerosis and diabetes, and for preventing peroxidation of cholesterole. If vitamin C is deficient, scurvy, subcutaneous bleeding, periodontitis, arthralgia, weight loss, anemia, etc. may be induced. Its overdose may induce diarrhea, nausea, abdominal distention, and kidney disease, which is still controversial. In case of its megadose up to 5,000 mg per day, no report for side effect was filed.

Ginkgetin is another essential component of the present invention, one of the five biflavons contained in the leaf or the fruit of Ginko Biloba, and has been traditionally used as a drug for anti-inflammation, anti-allergy, and anti-rheumatism drug. At present, biflavons and derivatives thereof have been known to have anti-cancer effect and analgesic effects, and to be stable analgesics which hardly cause cytotoxicity. Generally, flavonoid group including biflavon is most discovered in the leaf, and biflavon is one of twenty kinds of flavonoid glycosides. In particular, ginkgetin is one of seven biflavons, and has been known to have pharmacological effects such as arterioectasis, convulsion, osmotic phenomenon reduction, thrombolysis et al. Also, ginkgetin acts by indirect blood control effect of catecholamines or stimulation of prostaglandin, and has been known to play a role of antioxidant. Specifically, ginkgetin has been studied for use as arthritis treatment, skin anti-inflammatory agent, arthritis assist therapeutic agent, anticancer agent, cerebration promoter, and antifungal agent.

Another composition that can be used in the present invention is maltol, which is found abundantly in passionflower. Recently, maltol has been actively studied as one of the effective ingredients of steamed red ginseng. Maltol is used extensively as food addictives like flavor agents and stabilizers, and not soluble in water, included in chicory, cocoa, coffee, etc. In terms of pharmacological effect, maltol is reported to be effective for growth inhibition of cancer cell, dementia prevention, platelet aggregation, weight loss, blood lipid reduction, dermatosis reduction et al. in human or animal.

Another composition that be used in the present invention is the vitamin A. It exists in nature in the form of vitamin A or precursor thereof, and is contained in greenish-yellow vegetables, melons, cucumbers, potatoes, tomatoes, eggs, milk, liver oil et al. Carotinoid contained in greenish-yellow vegetables is a precursor of vitamin A. Vitamin A consists of three biogenic activation agents such as retinol, retinal and retino phosphoric acid. Alpha carotene, beta carotene, gamma carotene, cryptoxanthine, etc. are converted to vitamin A in the intestinal mucosal. About 90% of vitamin A stored in the body is in the liver, and the rest are found in the lung, hypodermic fat, kidney and the adrenal gland. Retinal among the activation agents of vitamin A is needed for the maintenance of normal eyesight, and relates to the regenerative function of animals. Retino phosphoric acid helps growth. Vitamin A has such pharmacological effects as eyesight maintenance, growth and differentiation of epithelial tissue, growth maintenance, maintenance of regenerative function, maintenance of biomembrane structure and function, retinal function control, anticancer effect, control effect of immunomechanism, etc. Deficiency of vitamin A leads to syndromes including nyctalopia induced by xerotic keratalgia and keratomalacia, conjunctival xerosis, xerotic keratalgia, necrotizing ulcer, secondary eye infection, vision loss et al., and toxicosis thereof is fatigue, malaise, headache, depilation, pain of muscle and bond, cerebral edema, vomiting, skin dryness, febricity, liver expansion, anemia et al.

Another composition that can be used in the present invention is Vitamin B1. It is contained in wheat, oat, peanut, vegetable, milk, various meat, brown rice, etc. Vitamin B1 relates to producing acetyl Co A by reacting with ATP in the body, activated in the form of TPP (thiamine pyrophosphate), and plays a major role in the metabolism of sugar, lipid, and amino acid, using acetyl Co A, and works as coenzyme in the conversion of pentose and hexose. Besides, it works as regulatory factor of nerve-muscle conduction, and phosphorylation thereof relates to axonal conduction for sodium channel. That is, vitamin B1 has a metabolism enhancing effect to energize carbohydrates absorbed into the body, and also works to normalize the heart function, the central nerves of the brain, and the peripheral nerves. Moreover, vitamin B1 is evenly distributed in the body tissues. Deficiency of vitamin B leads to syndromes including beriberi, anorexia, dyspepsia, ulcerative enteritis, chronic diarrhea, muscle fatigue, motor abnormality, nerve system disequilibrium et al.

Another composition that can be used in the present invention is the Vitamin D. It is effective for the absorption of calcium; the control of various factors related to destruction and regeneration of bone; the absorption increase of calcium and phosphorus in intestines; the secretion inhibition of parathyroid hormone; the differentiation promotion of osteoblast; the function inhibition of osteoclast by production inhibition of IL-1, IL-6, TNFα, etc.; the re-absorption promotion of calcium and phosphorus in kidney; etc.; and healthy persons who has exposure to the sun need not take it additionally. It has been reported that its toxicosis includes hypercalcemia, anorexia, nausea, vomiting, hydrodipsomania, frequent urination, muscle reduction, diarrhea, arthralgia, skeleton reduction et al. Deficiency of vitamin D leads to syndromes including gibbus, tooth decay, osteomalacia, juvenile ateliosis, myasthenia, agitation et al.

Another composition that can be used in the present invention is vitamin E. It requires bile acid when absorbed into the intestine, and enters the vascular system through the lymphatic system. Its absorption rate declines as the lipid and intake increase. Also, vitamin E is moved by lipoprotein in the blood, and is in the form of tocopherol alpha. It has an antioxidant effect, and prevents polyunsaturated fatty acid from being oxidized and destructed by free radicals. Its deficiency may lead to hemolysis phenomenon, and its toxicosis may cause headache, retching, fatigue, dizziness, blurred vision, change of epithelium tissue, and may increase the danger of bleeding for patients with vitamin K deficiency.

Another composition that can be used in the present invention is the Vitamin B. It is separated from folic acid and spinach, and is a complex of pteroyl monoglutamic acid, pterin cycling, para-aminobenzoic acid, and glutamic acid. It is stable under heat treatment, but easily destroyed in acid solutions and by reductants. And, it plays a major role with biosynthesis of funin, pyrimidine, and metabolism of serine-glycine. Vitamin B is important for hair health, skin, nerves, mucosa, and blood, and is effective for immunological functions, inhibition of arteriosclerosis, and mucosal cancer. Deficiency of vitamin B leads to syndromes including asthenia, depression, amnesia, megaloblastic anemia, weight loss, disorder of digestive system, growth failure, etc.

These essential ingredients and other optional elements partially enhance the immunity and inhibit certain viruses, but there have been no reports or discoveries relating to the complete prevention of cold using an appropriate combination of these compositions.

Thus, the present inventors have sought natural composition having optimal combination ratio which meets the above needs, and developed a new composition which has superior effects on cold virus inhibition and immunity enhancement, but does not show any cytotoxicity, comprising the above new ingredients whose anti-virus effects have not been known yet, as essential elements, and additionally vitamins. Therefore, they completed the present invention.

The composition for prevention of virus diseases according to the present invention has been shown to have superior effect, and to be used safely, in the clinical experiment and animal experiments, and can be prepared in various forms in view of convenience in preparation and characteristic of materials.

### DETAILED DESCRIPTION OF THE INVENTION

### Technical Subject

The object of the present invention is to provide a new composition comprising crude drug and vitamin as main ingredients. This new composition has excellent inhibition effect for cold viruses such as coronaviruses, rhinoviruses, adenoviruses and influenza viruses; does not have cytotoxicity, and can remarkably increase immunity. Also, this new composition can activate CTL by reacting with specific antigens, eliminate efficiently free oxygen radicals, and selectively kill malignant cells, by ideal combination of ingredients. Specifically, this composition is effective for killing cells infected with virus by increasing immune response to specific antigen, infectious cells by rapidly increasing CTL that is responsive to specific antigen of influenza virus, and infectious cells by activating Th1 CD4+ cell. Thus, this new composition is useful for preventing viral diseases such as cold by killing cells infected with virus and by activating the immune system.

Another object of the present invention is to provide health care products comprising the above composition.

Another object of the present invention is to provide use of the composition comprising vitamin C and ginkgetin for preparing medicine to prevent cold.

Another object of the present invention is to provide a method of preventing cold, comprising administering a prophylactically effective amount of vitamin C and ginkgetin.

Another object of the present invention is to provide a method of preparing cold prophylactic by mixing vitamin C and ginkgetin.

### Mode for Carrying out the Invention

To accomplish the above objects, the present invention provides a composition for preventing cold comprising vitamin C and ginkgetin.

Preferably, the composition of the present invention comprises 20 to 90% by weight of vitamin C, 1 to 20% by weight of ginkgetin, more preferably further including maltol.

Also, the composition of the present invention may further comprise one or more vitamins selected from vitamin A, vitamin B, vitamin B1, vitamin D, and vitamin E.

Preferably, one embodiment of the present invention comprises 20 to 90% by weight of vitamin C; 1 to 10% by weight of ginkgetin; 0.01 to 5% by weight of maltol; and 0.01 to 7% by weight of each of vitamins A, B1, E, D, and B.

The above composition ratios of the ingredients are based on repeated experiment results. If the ratios are lower than the lowest limits, physiological activation effect of these ingredients may decrease, and if they are higher than the highest limits, the synergy and interaction effects of these ingredients may decrease since physiological activation effects of other ingredients may decrease.

The present invention provides health care products comprising the above composition.

Also, the present invention provides use of the composition comprising vitamin C and ginkgetin for preparing medicine to prevent cold.

Moreover, the present invention provides a method for preventing cold comprising administering a prophylactically effective amount of vitamin C and ginkgetin to patients in need of prevention of cold.

Further, the present invention provides a method for preparing cold prophylactic by mixing vitamin C and ginkgetin.

The present invention is described in more detail below.

The composition of the present invention comprises vitamin C and ginkgetin as essential ingredients. As understood by the following experimental examples, some cold prevention effect is shown in the group administrating vitamin C, but administration of only vitamin C was not enough to completely prevent cold. However, administration of vitamin C and ginkgetin at the same time could almost completely prevent cold. By now, it is not known that cold can be prevented by administrating ginkgetin. Thus, the present invention shows for the first time that cold can be efficiently prevented by combined administration of vitamin C and ginkgetin.

Ginkgetin used in the composition of the present invention is extracted according to conventional methods comprising boiling water extraction method and sonication method, etc., by using solvent such as water or alcohol, from leaves and fruits of gingko trees, and is powdered according to conventional refining-process and/or lyophilization.

Also, as shown in the following experimental examples, the cold prevention effect was particularly good in the group further comprising maltol, and the cold prevention effect was 100% in the group additionally comprising vitamin.

The composition of the present invention can be prepared according to conventional methods in the pharmaceutical field. That is, it can be prepared into conventional preparations, for example, drinks such as solution, syrup and capsule, mixed with pharmaceutically acceptable carrier, excipient, etc.; and can be administered orally or parenterally. Preferably, the composition of the present invention may be orally administered in capsules or drinks before and/or after the meal.

Preferably, capsule and solution comprising the composition of the present invention may be used as health care products. Here, “health care products” mean food products prepared and processed in the form of tablet, capsule, powder, granule, solution, pill, etc., by using material or ingredients having useful function to the human body.

The composition of the present invention is appropriately selected according to the extent of absorption of active ingredients into the body; excretion rate; age, weight, sex, and condition of patient; severity of treated disease, etc., but generally, it is preferable to administer the present composition in 0.01˜500 g/kg a day in adult, and more preferable in 0.1˜200 mg/kg. So formulated unitary dosage preparation may be administered several times at regular intervals, if necessary.

Below, the present invention is explained in more detail by the following Examples, but it should be understood that they are not intended to construe the present invention in any restricted way.

### PREPARATION OF EXAMPLES 1˜8

Examples 1˜8 according to the present invention are prepared by mixing vitamin C, ginkgetin, maltol, and other vitamins in the composition ratios of the following Table 1. The unit of each ingredient means the weight % of each ingredient in the total composition.

TABLE 1

| | Vitamin | | | Vitamin | Vitamin | Vitamin | Vitamin | Vitamin |
|---|---|---|---|---|---|---|---|---|
| | C | Ginkgetin | Maltol | A | B1 | E | D | B |
| Example 1 | 90 | 10 | — | — | — | — | — | — |
| Example 2 | 95 | 5 | — | — | — | — | — | — |
| Example 3 | 89 | 10 | 1 | — | — | — | — | — |
| Example 4 | 90 | 7 | 3 | — | — | — | — | — |
| Example 5 | 78 | 5 | 3 | 7 | — | — | — | 7 |
| Example 6 | 75 | 10 | 1 | — | — | — | 7 | 7 |
| Example 7 | 80 | 4 | 2 | 7 | 7 | — | — | — |
| Example 8 | 69 | 10 | 1 | 4 | 4 | 4 | 4 | 4 |

### FORMULATION EXAMPLE 1

### Preparation of Capsule

| | | |
|---|---|---|
| Composition of Example 1 | 100 | mg |
| Lactose | 100 | mg |
| Starch | 93 | mg |
| Talc | 2 | mg |

| | |
|---|---|
| Stearinic acid magnesium | q.s. |

The above-mentioned ingredients are mixed, and filled in gelatin capsule according to conventional preparation method to give capsule.

### FORMULATION EXAMPLE 2

### Preparation of Capsule

| | | |
|---|---|---|
| Composition of Example 3 | 200 | mg |
| Lactose | 100 | mg |
| Starch | 93 | mg |
| Talc | 2 | mg |

| | |
|---|---|
| Stearinic acid magnesium | q.s. |

The above-mentioned ingredients are mixed, and filled in gelatin capsule according to conventional preparation method to give capsule.

### FORMULATION EXAMPLE 3

### Preparation of Capsule

| | | |
|---|---|---|
| Composition of Example 8 | 200 | mg |
| Lactose | 100 | mg |
| Starch | 93 | mg |
| Talc | 2 | mg |

| | |
|---|---|
| Stearinic acid magnesium | q.s. |

The above-mentioned ingredients are mixed, and filled in gelatin capsule according to conventional preparation method to give capsule.

### FORMULATION EXAMPLE 4

### Preparation of Solution

| Composition of Example 4 | 300 | mg |
|---|---|---|
| Sucrose | 20 | g |
| Isomerized sugar | 20 | g |
| Smell of lemon | q.s. | |
| Total amount after adding purified water | 100 | ml |

The above-mentioned ingredients are mixed according to conventional preparation method for solution, filled in a brown bottle of 100 ml, and sterilized to give solution.

### FORMULATION EXAMPLE 5

### Preparation of Solution

| Composition of Example 8 | 300 | mg |
|---|---|---|
| Sucrose | 20 | g |
| Isomerized sugar | 20 | g |
| Smell of lemon | q.s. | |
| Total amount after adding purified water | 100 | ml |

The above-mentioned ingredients are mixed according to conventional preparation method for solution, filled in a brown bottle of 100 ml, and sterilized to give solution.

### FORMULATION EXAMPLE 6

### Preparation of Drink

After preparing a composition comprising 10% by weight of the composition of Example 8, 0.1% by weight of citric acid, 0.05% by weight of food color additive, 0.05% by weight of orange essence, and 5.0% by weight of fructose, and further general functional beverage base, purified water is added thereto to give drink.

### EXPERIMENTAL EXAMPLE 1

Efficacy of the present composition (the composition of Example 1) and placebo were tested on 15 persons of the experiment group and 15 persons of the control group by double blinded placebo test for 40 months from 2000˜2003. Both the composition of Example 1 according to the present invention and placebo were administered three times a day 1 g per administration after meal. Here, placebo was prepared with wheat flour. The experimental results were shown in the following Tables 2 and 3. In the experiment group taking the composition of the present invention, the frequency of cold occurrence remarkably decreased throughout 48 months as shown in the following Table 2, and the control group shows total 162 cases of cold, that is, an average of 2.7 times a year for each person. Here, ‘cold’ is defined by the symptoms of high fever or slight fever, sore throat, dry cough, headache, myalgia, and chill.

TABLE 2
Frequency of cold in the experiment group

| | Age | Sex | Frequency of cold |
|---|---|---|---|
| 1 | 62 | Man | 1 |
| 2 | 59 | Man | 2 |
| 3 | 61 | Man | 2 |
| 4 | 71 | Woman | 2 |
| 5 | 58 | Man | 0 |
| 6 | 39 | Woman | 0 |
| 7 | 43 | Woman | 0 |
| 8 | 45 | Man | 1 |
| 9 | 68 | Woman | 0 |
| 10 | 78 | Man | 0 |
| 11 | 45 | Man | 1 |
| 12 | 52 | Man | 0 |
| 13 | 63 | Woman | 0 |
| 14 | 49 | Woman | 1 |
| 15 | 81 | Man | 1 |

TABLE 3
Frequency of cold in the control group

| | Age | Sex | Frequency of cold |
|---|---|---|---|
| 1 | 45 | Man | 11 |
| 2 | 61 | Woman | 12 |
| 3 | 53 | Man | 9 |
| 4 | 49 | Man | 10 |
| 5 | 63 | Woman | 10 |
| 6 | 38 | Man | 12 |
| 7 | 64 | Man | 8 |
| 8 | 57 | Woman | 11 |
| 9 | 71 | Woman | 12 |
| 10 | 63 | Man | 10 |
| 11 | 48 | Man | 13 |
| 12 | 58 | Man | 12 |
| 13 | 69 | Woman | 9 |
| 14 | 63 | Man | 11 |
| 15 | 60 | Man | 12 |

### EXPERIMENTAL EXAMPLE 2

The present experiment example analyzed cold cases of 12 persons in the experiment group and 9 persons in the control group for 40 months from 1999 to 2003. The experiment group took the composition of Example 3 three times a day 1 g per administration after meal, but the control group took nothing. Here, ‘cold’ is defined to show high fever or slight fever, sore throat, dry cough, headache, myalgia, and chill. The experimental results are shown in the following Tables 4 and 5. In the experiment group, only one case of cold was reported, but the case was found that the subject stopped taking the composition. In the control group, total 123 cases of cold, that is, an average of 3.5 times a year for one person, were reported.

TABLE 4
Frequency of cold in the experiment group

| | Age | Sex | Frequency of cold |
|---|---|---|---|
| 1 | 63 | Man | 0 |
| 2 | 65 | Man | 0 |
| 3 | 66 | Woman | 0 |
| 4 | 78 | Man | 0 |
| 5 | 61 | Man | 0 |
| 6 | 63 | Man | 0 |
| 7 | 64 | Man | 0 |
| 8 | 38 | Man | 0 |
| 9 | 59 | Woman | 0 |
| 10 | 64 | Man | 1 |
| 11 | 65 | Man | 0 |
| 12 | 72 | Man | 0 |

TABLE 5
Frequency of cold in the control group

| | Age | Sex | Frequency of cold |
|---|---|---|---|
| 1 | 43 | Woman | 12 |
| 2 | 47 | Woman | 11 |
| 3 | 39 | Man | 13 |
| 4 | 44 | Man | 14 |
| 5 | 42 | Woman | 12 |
| 6 | 46 | Woman | 14 |
| 7 | 39 | Man | 15 |
| 8 | 38 | Man | 12 |
| 9 | 66 | Man | 10 |

### EXPERIMENTAL EXAMPLE 3

The present experiment example analyzed cold cases in the experiment group who took the composition of Example 8, and the control groups I and II who took vitamin C and placebo (wheat flour) respectively, throughout 40 months from 1999 to 2003. The result is shown in the following Tables 6, 7 and 8. The intake is the same as 1 g after meal. Here, ‘cold’ is defined to show high fever or slight fever, sore throat, dry cough, headache, myalgia, and chill.

TABLE 6
Frequency of cold in the experiment group

| | Age | Sex | Frequency of cold |
|---|---|---|---|
| 1 | 62 | Man | 0 |
| 2 | 59 | Man | 0 |
| 3 | 61 | Man | 0 |
| 4 | 71 | Woman | 0 |
| 5 | 58 | Man | 0 |
| 6 | 39 | Woman | 0 |
| 7 | 43 | Woman | 0 |
| 8 | 45 | Man | 0 |
| 9 | 68 | Woman | 0 |
| 10 | 78 | Man | 0 |
| 11 | 45 | Man | 0 |
| 12 | 52 | Man | 0 |
| 13 | 63 | Woman | 0 |
| 14 | 49 | Woman | 0 |
| 15 | 81 | Man | 0 |

TABLE 7
Frequency of cold in the control group taking vitamin C

| | Age | Sex | Frequency of cold |
|---|---|---|---|
| 1 | 59 | Man | 6 |
| 2 | 62 | Woman | 4 |
| 3 | 58 | Woman | 4 |
| 4 | 63 | Man | 5 |
| 5 | 48 | Man | 6 |
| 6 | 46 | Woman | 4 |
| 7 | 63 | Woman | 5 |
| 8 | 58 | Man | 4 |
| 9 | 49 | Woman | 4 |
| 10 | 44 | Man | 5 |
| 11 | 63 | Woman | 5 |
| 12 | 71 | Man | 4 |
| 13 | 68 | Woman | 7 |
| 14 | 57 | Man | 4 |
| 15 | 63 | Man | 5 |

TABLE 8
Frequency of cold in the control group taking placebo

| | Age | Sex | Frequency of cold |
|---|---|---|---|
| 1 | 45 | Man | 11 |
| 2 | 61 | Woman | 12 |
| 3 | 53 | Man | 9 |
| 4 | 49 | Man | 10 |
| 5 | 63 | Woman | 10 |
| 6 | 38 | Man | 12 |
| 7 | 64 | Man | 8 |
| 8 | 57 | Woman | 11 |
| 9 | 71 | Woman | 12 |
| 10 | 63 | Man | 10 |
| 11 | 48 | Man | 13 |
| 12 | 58 | Man | 12 |
| 13 | 69 | Woman | 9 |
| 14 | 63 | Man | 11 |
| 15 | 60 | Woman | 12 |

As a result of the above experiment, the average of cold case a year per person in the experiment group who took the present composition, the control group I who took vitamin C, and the control group II who took placebo was 0, 1.5, and 5.6, respectively, all of which are statistically significant difference under 0.05% of significance probability. In sum, the composition of the present invention accomplished 100% of cold prevention effect.

### EXPERIMENTAL EXAMPLE 4

The object of the present experiment is to show immunity improving effect of the composition of Example 1 of the present invention by measuring cytokines, a kind of growth factor such as IL-2, INF-γ, IL-4, IL-10 and IL-12. First, 96-well plate was coated with 4 pg/ml of each antibody at 4° C. for 18 hrs, washed with distilled water containing 0.05% of tween 20, and blocked at 37° C. for one hour with 1% of BSA and 0.05% of tween. Then, the experiment sample and standard sample of 100 ml were filled in each well, and cultivated at 37° C. for 2 hours. After such cultivating, the plates were washed, biotynilated secondary antibody was filled in each plate by 0.75 ml per ml, and streptavidin ALP was added thereto, which was cultivated at 37° C. for 30 minutes. After washing the plates again, alkaline phosphatase PNPP was added thereto, and the plates were cultivated for 30 minutes and measured with ELISA reader at 405 nm. Each sample was collected from those who took placebo, vitamin C, and the composition of the present invention for 40 months.

TABLE 9
Effect of the present composition for protective
immunity increase (immune response of Th1)

| | Number | | | | | |
|---|---|---|---|---|---|---|
| | of the | | | | | |
| Medicine | experiment | Cytokine in serum (pg/ml) | | | | |
| administrated | sample | IL-2 | INF-γ | IL-4 | IL-10 | IL-12 |
| Placebo | 10 | 31 ± 16 | 36 ± 9 | 0.16 ± 0.03 | 12 ± 9 | 11 ± 6 |
| The present | 10 | 63 ± 21 | 118 ± 16 | 0.12 ± 0.02 | 8 ± 7 | 38 ± 11 |
| composition | | | | | | |
| Vitamin C | 10 | 51 ± 19 | 68 ± 12 | 0.14 ± 0.03 | 10 ± 8 | 20 ± 7 |

This result shows that immune response of Th1 increased very significantly in the experimental group who took the composition of the present invention. In particular, in case of INF-γ and IL-12 levels, the experimental group who took the composition of the present invention and the control groups who took placebo and vitamin C show very significant difference under significance probability of 0.005 and below. However, in case of IL-4 and IL-10 levels which are criterion of immune response of Th2, the two groups do not show significant difference under significance probability of 0.09 and below. This experiment result shows that cold and influenza are prevented, and symptoms thereof are relieved for increasing immune response of Th1 by taking the composition of the present invention.

### INDUSTRIAL APPLICABILITY

The composition of the present invention has excellent effect for inhibiting cold viruses such as coronaviruses, rhinoviruses, adenoviruses, and influenza viruses, and so cold can be completely prevented by the present composition. Also, no side effect is shown in administering the present composition for 40 months, and so the composition of the present invention may be used as health care products or medicines.

1. A composition for preventing cold comprising vitamin C and ginkgetin.

2. The composition according to claim 1, comprising 20 to 90% by weight of vitamin C; and 1 to 20% by weight of ginkgetin.

3. The composition according to claim 1, further comprising maltol.

4. The composition according to claim 1, further comprising more than one kind of vitamins selected from vitamin A, vitamin B, vitamin B1, vitamin D, and vitamin E.

5. The composition according to claim 4, comprising 20 to 90% by weight of vitamin C; 1 to 10% by weight of ginkgetin; 0.01 to 5% by weight of maltol; and 0.01 to 7% by weight of each of vitamin A, B1, E, D, and B.

6. (canceled)

7. Use of a composition comprising vitamin C and ginkgetin to prepare a medicine for cold prevention.

8. Use of the composition according to claim 7, comprising 20 to 90% by weight of vitamin C; and 1 to 20% by weight of ginkgetin.

9. Use of the composition according to claim 7, further comprising maltol.

10. Use of the composition according to claim 7, further comprising more than one kind of vitamins selected from vitamin A, vitamin B, vitamin B1, vitamin D, and vitamin E.

11. Use of the composition according to claim 10, comprising 20 to 90% by weight of vitamin C; 1 to 10% by weight of ginkgetin; 0.01 to 5% by weight of maltol; and 0.01 to 7% by weight of each of vitamin A, B1, E, D, and B.

12. A method of preventing cold, comprising administering a prophylactically effective amount of vitamin C and ginkgetin to patients in need of prevention of cold.

13. The method according to claim 12, comprising administering 20 to 90% by weight of vitamin C; and 1 to 20% by weight of ginkgetin.

14. The method according to claim 12, comprising further administering maltol.

15. The method according to claim 12, comprising further administering more than one kind of vitamins selected from vitamin A, vitamin B, vitamin B1, vitamin D, and vitamin E.

16. The method according to claim 15, comprising administering 20 to 90% by weight of vitamin C; 1 to 10% by weight of ginkgetin; 0.01 to 5% by weight of maltol and 0.01 to 7% by weight of each of vitamin A, B1, E, D, and B.

17. A method for preparing cold prophylactic by mixing vitamin C and ginkgetin.

18. The method according to claim 17, comprising mixing 20 to 90% by weight of vitamin C; and 1 to 20% by weight of ginkgetin.

19. The method according to claim 17, comprising further mixing maltol.

20. The method according to claim 17, comprising further mixing more than one kind of vitamins selected from vitamin A, vitamin B, vitamin B1, vitamin D, and vitamin E.

21. The method according to claim 19, comprising mixing 20 to 90% by weight of vitamin C; 1 to 10% by weight of ginkgetin; 0.01 to 5% by weight of maltol; and 0.01 to 7% by weight of each of vitamin A, B1, E, D, and B.
